(19) United States
(12) Patent Application Publication
(10) Pub. No.: US 2023/0414691 A1
(43) Pub. Date: Dec. 28, 2023

(54) COMPOSITION INCLUDING FRACTION OF SYZYGIUM FORMOSUM EXTRACT AS ACTIVE INGREDIENT

(71) Applicant: CARBOEXPERT INC., Daejeon (KR)

(72) Inventors: Chang Kyu LEE, Daejeon (KR); Nan Young LEE, Daejeon (KR); Hyunah PARK, Daejeon (KR); Thi Minh Nguyet NGUYEN, Daejeon (KR)

(73) Assignee: CARBOEXPERT INC., Daejeon (KR)

(21) Appl. No.: 18/039,255

(22) PCT Filed: Sep. 7, 2021
(86) PCT No.: PCT/KR2021/012150

§ 371 Date: May 29, 2023

(30) Foreign Application Priority Data
Nov. 27, 2020 (KR) 10-2020-0162783

(51) Int. Cl. A61K 36/61; A61K 47/32; A61K 47/10; A61P 29/00; A61K 47/36; A61K 47/44; A61K 47/14

(52) U.S. Cl. CPC A61K 36/61; A61K 47/32; A61K 47/10; A61P 29/00; A61K 47/36; A61K 47/44; A61K 47/14; A61K 2236/39

(57) ABSTRACT

Provided is a composition including a fraction of the Syzygium formosum extract as an active ingredient, which inhibits the expression of inflammatory cytokines to prevent, ameliorate, or treat inflammation disease, and when the composition is included in cosmetics, the prepared cosmetics may have high stability.

### CROSS-REFERENCE TO RELATED APPLICATION

This application is based on and claims priority under 35 U.S.C. § 119 to Korean Patent Application No. 10-2020-0162783, filed on Nov. 27, 2020, in the Korean Intellectual Property Office, the disclosure of which is incorporated by reference herein in its entirety.

### BACKGROUND

### 1. Field

One or more embodiments relate to a composition including a fraction of Syzygium formosum extract as an active ingredient.

### 2. Description of the Related Art

Inflammation is a local protective reaction of the living body in response to physical trauma, infection by harmful chemicals, bacteria, fungi, and viruses, or pathological conditions caused by irritants in in-vivo metabolites. Inflammation is triggered by various inflammatory mediators produced from damaged tissues and migrating cells. In an inflammatory reaction, plasma is accumulated in an inflammatory site to dilute the toxicity secreted by bacteria, blood flow increases, and symptoms such as erythema, pain, edema, and heating are accompanied. In normal cases, the living body neutralizes or removes pathogenic factors through an inflammatory reaction and regenerates damaged tissues to restore normal structure and function, but otherwise, the inflammatory reaction may progress to disease states such as chronic inflammation.

Macrophages are the main cells responsible for innate immunity, and are activated by numerous factors such as cytokines and bacterial lipopolysaccharide (LPS). Activated macrophages produce pro-inflammatory cytokines such as tumor necrosis factor-α (TNF-α), interleukin-6 (IL-6), and interleukin-1 (IL-1), as well as inflammatory factors, such as nitric oxide (NO) and prostaglandin E2 (PGE2).

In addition, cyclooxygenase (COX) has the function of COX and the activity of hydroperoxidase (HOX) and synthesizes intermediates PGG₂ and PGG₂ from arachidonic acid, and, produces, using these compounds, PGE₂, PGF₂, PGD₂, prostacycline, and thromboxane A2 (TxA2). Of the two isoforms of COX, COX-2 plays an important role in causing an inflammatory reaction by rapidly inducing its expression during an inflammatory reaction and producing PGE₂ or the like (Weisz A., Biochem. J., 316:209-215, 1996; (Miller M. J. et al., Mediators of inflammation, 4:387-396, 1995: Appleton L. et al., Adv. Pharmacol., 35:27-28, 1996). PGE₂ not only plays a role as a mediator of the known inflammatory response, but also inhibits the production of inflammatory cytokines such as TNF-α, IL-1β, IL-8, and IL-12 in macrophages.

Syzygium formosum is an evergreen tree that lives in the Southeast Asia countries such as Bangladesh, India, Myanmar, Thailand, Laos, and Vietnam, and grows to a height of 10 m. In Vietnam and Laos, Syzygium formosum is cultivated and the fruit of this tree is used for food.

However, it has not been disclosed that the fraction of Syzygium formosum extract as in the present disclosure is effective in inhibiting inflammation.

### SUMMARY

One aspect is to provide a cosmetic composition including a fraction of the Syzygium formosum extract as an active ingredient.

Another aspect is to provide a composition for external application for skin, including a fraction of the Syzygium formosum extract as an active ingredient, for ameliorating skin inflammation.

Another aspect is to provide a health functional food for ameliorating skin inflammation, the health functional food including a fraction of the Syzygium formosum extract as an active ingredient.

Another aspect is to provide a pharmaceutical composition for the prevention or treatment of skin inflammatory diseases, the pharmaceutical composition including a fraction of the Syzygium formosum extract as an active ingredient.

Additional aspects will be set forth in part in the description which follows and, in part, will be apparent from the description, or may be learned by practice of the presented embodiments of the disclosure.

One aspect is to provide a cosmetic composition including a fraction of the Syzygium formosum extract as an active ingredient.

The Syzygium formosum may be at least one selected from whole, root, stem, branch, leaf, seed, or fruit. In an embodiment, the Syzygium formosum extract may be obtained by extraction from leaves of Syzygium formosum.

The term “active ingredient” or “effective amount” used herein refers to an amount of a composition used in the practice of the disclosure provided herein sufficient to alleviate, inhibit or prevent progression of a disease, disorder or condition, or at least one symptom thereof.

The term “fraction” used herein may refer to a result obtained by performing fractioning to isolate a particular component or a particular component group from a mixture including various components.

The fractionation method for obtaining the fraction is not particularly limited, and may be performed according to a method commonly used in the art.

As a non-limiting example of the fractionation method, there is a method of obtaining a fraction from the extract by treating, with a solvent, the extract obtained by extracting Syzygium formosum.

The kind of fractionation solvent used to obtain the fraction is not particularly limited, and any solvent known in the art may be used therefor. Non-limiting examples of the fractionation solvent include polar solvents such as water and alcohols such as butanol, and nonpolar solvents, such as hexane, ethyl acetate, chloroform, and dichloromethane. These may be used alone or in combination of two or more. When an alcohol is used as the fractionation solvent, a C1 to C4 alcohol may be used.

The fraction of the Syzygium formosum extract may be a hexane fraction, an ethyl acetate fraction, a butanol fraction or a water fraction of the Syzygium formosum extract, and these fractions may be obtained by repeatedly performing the fractioning once to 5 times, 2 times to 5 times, 3 times to 5 times, or 2 times to 4 times. In the fraction according to an embodiment, the fraction may be a water fraction, and in this case, the fractioning may be performed three times.

The fractioning may include adding, to the Syzygium formosum extract, the fractionation solvent in 1 to 30 (vol/weight) times, 5 to 30 (vol/weight) times, 5 to 20 (vol/weight) times, 10 to 30 times (vole/weight), or 10 to 20 times (vole/weight) greater than that of the Syzygium formosum extract. For example, the fractionation solvent may be added in an amount of 100 ml to 3000 ml to 100 g of the Syzygium formosum extract.

Compared to the Syzygium formosum extract, the fraction of the Syzygium formosum extract may include a high concentration of triterpene compounds such as asiatic acid, madecassic acid, colosolic acid, and maslinic acid, betulinic acid, ursolic acid, or oleanolic acid. Accordingly, the fraction of the Syzygium formosum extract may have higher anti-inflammation, anti-allergic or skin regeneration effects than the Syzygium formosum extract. Accordingly, the cosmetic composition may be for ameliorating skin inflammation.

The term “skin inflammation” used herein refers to a disease caused by inflammation-inducing substances (inflammatory cytokines) such as Interleukin-6 (IL-6 ATP) or tumor necrosis factor-α (TNF-α) secreted from immune cells, such as macrophages, by excessively antagonizing the human immune system due to harmful stimuli such as inflammation-inducing factors or irradiation of radiation. The composition according to an embodiment has anti-inflammatory, anti-allergic or skin regeneration effects by reducing the activity of inflammatory cytokines.

In an embodiment, the cosmetic composition may further include an emollient, a humectant, and a thickener.

The emollient may be selected from cetearyl alcohol, cetyl palmitate, beeswax, squalane, cetylethylhexanoate and caprylic/capric triglyceride, the humectant may be selected from 1,2-hexanediol, dipropylene glycol, and glycerin, and the thickener may be selected from xanthan gum and ammonium acryloyldimethyltaurate/VP copolymer.

The Syzygium formosum extract includes a large amount of plant metabolites such as amino acids and sugars, as well as a triterpene-based compound, and accordingly, cosmetics including the same may have color change, viscosity instability, and emulsion stability. In contrast, the fraction of the Syzygium formosum extract contains a triterpene-based compound at a high concentration, and thus, cosmetics including the same may have little color change and stable viscosity, resulting in higher stability.

The composition may include the fraction of the Syzygium formosum extract, based on the total weight of the composition, in an amount of about 0.001 wt % to about 80 wt %, for example, about 0.01 wt % to about 60 wt %, about 0.01 wt % to about 40 wt %, about 0.01 wt % to about 30 wt %, about 0.01 wt % to about 20 wt %, about 0.01 wt % to about 10 wt %, about 0.01 wt % to about 5 wt %, about 0.05 wt % to about 60 wt %, about 0.05 wt % to about 40 wt %, about 0.05 wt % to about 30 wt %, about 0.05 wt % to about 20 wt %, about 0.05 wt % to about 10 wt %, about 0.05 wt % to about 5 wt %, about 0.1 wt % to about 60 wt %, about 0.1 wt % to about 40 wt %, about 0.1 wt % to about 30 wt %, about 0.1 wt % to about 20 wt %, about 0.1 wt % to about 10 wt %, or about 0.1 wt % to about 5 wt %.

The cosmetic composition may have a formulation selected from a softening lotion, nourishing lotion, nourishing cream, moisture cream, massage cream, essence, ampoule, gel, eye cream, cleansing cream, cleansing foam, cleansing water, pack, spray, powder, lotion and ointment. The cosmetic composition may additionally include at least one of cosmetically acceptable carriers which are mixed in general skin cosmetics, and for use as such cosmetically acceptable carriers, for example, oil, water, surfactants, moisturizers, lower alcohols, thickeners, chelating agents, pigments, preservatives, fragrances, and the like may be appropriately mixed. However, embodiments of the present disclosure are not limited thereto.

Another aspect is to provide a composition for external application for skin, including a fraction of the Syzygium formosum extract as an active ingredient, for ameliorating skin inflammation.

The external application for skin may be a cream, a gel, an ointment, a skin emulsifier, a skin suspension, a transdermal delivery patch, a drug-containing bandage, a lotion, or a combination thereof.

For the external application for skin, components that are commonly used in external applications for skin such as cosmetics and pharmaceuticals, for example, aqueous components, oily components, powder components, alcohols, moisturizers, thickeners, ultraviolet absorbers, whitening agents, preservatives, antioxidants, surfactants, fragrances, colorants, various skin nutrients, etc. may be appropriately mixed as needed.

For the external application for skin, metal sequestering agents such as disodium edetate, trisodium edetate, sodium citrate, sodium polyphosphate, sodium metaphosphate, and gluconic acid, caffeine, tannin, bellapamil, licorice extract, glabridin, hot water extract of fruit, such as caline, herbal medicines, drugs such as tocopherol acetate, glytilithinic acid, tranexamic acid and derivatives or salts thereof, vitamin C, magnesium ascorbic acid phosphate, glucoside ascorbic acid, arbutin, kojic acid, sugars such as glucose, fructose, trehalose, etc, may also be appropriately mixed.

Another aspect is to provide a health functional food for ameliorating skin inflammation, the health functional food including a fraction of the Syzygium formosum extract as an active ingredient.

The health functional food is a food that is manufactured using nutrients that are liable to be deficient in daily meals or raw materials or components that have useful functions for the human body (hereinafter, referred to as ‘functional raw materials’), and may be any food that helps maintain health or prevent and/or ameliorate certain diseases or symptoms. However, there are no restrictions on the final product form thereof. For example, the health functional food may have a formulation selected from powders, granules, tablets, capsules, pills, gels, jelly, suspensions, emulsions, syrups, tea bags, leached tea, or health beverages.

The amount of the active ingredient (i.e., the fraction of the Syzygium formosum extract) contained in the health functional food is not particularly limited and may vary appropriately depending on the form of the food and the target use. For example, the amount of the active ingredient may be from about 0.1 wt % to about 50 wt % based on the total weight of the food.

The health functional foods may additionally include at least one selected from nutrients, vitamins, minerals (electrolytes), flavoring agents such as synthetic flavoring agents or natural flavoring agents, coloring agents, flavor enhancers (cheese, chocolate, etc.), pectic acid or a salt thereof, alginic acid or a salt thereof, organic acids, protective colloidal thickeners, pH adjusters, stabilizers, preservatives, glycerin, alcohols, and carbonates used in carbonated beverages. The amount of these additives may be from about 0.001 parts by weight to about 20 parts by weight per 100 parts by weight of the health functional food, but is not limited thereto.

Another aspect is to provide a pharmaceutical composition for the prevention or treatment of skin inflammatory diseases, the pharmaceutical composition including a fraction of the Syzygium formosum extract as an active ingredient.

The skin inflammatory disease may be selected from skin wounds, dermatitis, atopic dermatitis, pruritus, eczema skin disease, dry eczema, erythema, urticaria, psoriasis, weak rash, and acne.

The term “prevention” used herein refers to partially or completely delaying or preventing the onset or recurrence of a disease, a disorder, or a concomitant symptom thereof, preventing the acquisition or reacquisition of a disease or a disorder, or reducing the risk of acquisition of a disease or a disorder. For example, the prevention refers to any action of inhibiting or delaying the occurrence of inflammation or inflammation-related diseases, disorders, or symptoms by administration of the composition according to the present disclosure.

The term “ameliorating” used herein may refer to any action that alleviates a condition or at least reduces a parameter related to a treatment, for example, the degree of a symptom.

The term “treatment” used herein includes alleviation or amelioration of pathological symptoms, reduction of the site of the disease, delaying or alleviating the progression of the disease, ameliorating, alleviating or stabilizing the disease state or symptom, partial or complete recovery, prolonging survival, and other beneficial treatment results.

The term “pharmaceutical composition” used herein may refer to a molecule or compound that imparts several beneficial effects upon administration to a subject. The beneficial effect may include enabling diagnostic decisions; ameliorating a disease, a symptom, a disorder, or a condition; reducing or preventing the onset of a disease, a symptom, a disorder, or a condition; and responding to a disease, a symptom, a disorder, or a condition.

The pharmaceutical composition may be administered parenterally during clinical administration and may be used in the form of a general pharmaceutical formulation. Parenteral administration may refer to administration through a route other than an oral route, for example, administration through a rectal route, an intravenous route, a peritoneal route, a muscle route, an arterial route, a transdermal route, a nasal route, an inhalation route, an ocular route, or a subcutaneous route. When the pharmaceutical composition of the present disclosure is used as a pharmaceutical product, at least one active ingredient showing the same or similar function may be additionally used.

The pharmaceutical composition may be in the form of a solution, suspension, syrup, or emulsion in an aqueous or oily medium, or may be formulated in the form of powder, granule, tablet, or capsule, and, for the formulation, may additionally include a dispersant or a stabilizer. When formulating the pharmaceutical composition, the pharmaceutical composition may be prepared using diluents or excipients such as fillers, extenders, binders, humectants, disintegrants, and surfactants. Preparations for parenteral administration may include sterilized aqueous solutions, non-aqueous solutions, suspensions, emulsions, lyophilized preparations, and suppositories. As the non-aqueous solvent and the suspension solvent, propylene glycol, polyethylene glycol, vegetable oil such as olive oil, and injectable ester such as ethyl oleate may be used. As a base for the suppository, Witepsol, Macrogol, Tween 61, cacao butter, ryurinji, glycerogelatin, and the like may be used.

The pharmaceutical composition may be used in combination with various carriers which are allowed for use in medication, such as physiological saline or organic solvents, and, to increase stability or absorption, carbohydrates such as glucose, sucrose or dextran, antioxidants such as ascorbic acid or glutathione, chelating agents, small molecule proteins, or other stabilizers may be used for use in medication.

In addition, the pharmaceutically effective amount and effective dosage of the pharmaceutical composition may vary depending on the formulation method, mode of administration, administration time, and/or route of administration of the pharmaceutical composition. In addition, the pharmaceutically effective amount and effective dosage of the pharmaceutical composition may vary depending on various factors and similar factors known in the medical field, for example, the type and degree of reaction to be achieved by the administration of the pharmaceutical composition, the type, age, weight, and general health condition of the subject to be administered, symptoms or seriousness of disease, gender, diet, excretion, or components of other drug compositions which are used together in a corresponding subject at the same time or different timings. Those of ordinary skill in the art may readily determine and prescribe an effective dosage for the target treatment. The administration of the pharmaceutical composition according to the present disclosure may be carried out once a day, or several times a day. Therefore, the above dosage does not limit the scope of the present disclosure in any aspects. The dosage of the pharmaceutical composition may be 1 ug/kg/day to 1,000 mg/kg/day per day.

The subject may be a mammal, for example a human, cow, a horse, a pig, a dog, sheep, a goat, or a cat. The subject may be an entity in need of healing of skin inflammation.

The terms and methods described for the above disclosure are applied equally to respective disclosures.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a graph showing cytotoxicity: FIG. 1A is a graph showing the cytotoxicity of Syzygium formosum extract, and of FIG. 1B is a graph showing the cytotoxicity of a fraction of the Syzygium formosum extract;

FIG. 2 is a graph showing the expression of COX-2 after UVB irradiation: FIG. 2A is a graph showing the expression of COX-2 after UVB irradiation followed by the treatment with Syzygium formosum extract, and FIG. 2B is a graph showing the expression of COX-2 after UVB irradiation followed by the treatment with the fraction of the Syzygium formosum extract;

FIG. 3 is a graph showing the expression of IL-1β after UVB irradiation: FIG. 3A is a graph showing the expression of IL-1β after UVB irradiation followed by the treatment with Syzygium formosum extract, and FIG. 3B is a graph showing the expression of IL-1β after UVB irradiation followed by the treatment with the fraction of the Syzygium formosum extract;

FIG. 4 is a graph showing the expression of IL-6 after UVB irradiation: FIG. 4A is a graph showing the expression of IL-6 after UVB irradiation followed by the treatment with Syzygium formosum extract, and FIG. 4B is a graph showing the expression of IL-6 after UVB irradiation followed by the treatment with the fraction of the Syzygium formosum extract;

FIG. 5 is a graph showing the expression of IL-8 after UVB irradiation: FIG. 5A is a graph showing the expression of IL-8 after UVB irradiation followed by the treatment with Syzygium formosum extract, and FIG. 5B is a graph showing the expression of IL-8 after UVB irradiation followed by the treatment with the fraction of the Syzygium formosum extract;

FIG. 6 is a graph showing the expression of TNF-α after UVB irradiation: FIG. 6A is a graph showing the expression of TNF-α after UVB irradiation followed by the treatment with Syzygium formosum extract, and FIG. 6B is a graph showing the expression of TNF-α after UVB irradiation followed by the treatment with the fraction of the Syzygium formosum extract;

FIG. 7 is a graph comparing the expression of IL-6 after UVB irradiation, followed by treatment with Centella asiatica extract, Syzygium formosum extract, or a fraction of the Syzygium formosum extract;

FIG. 8 is a graph comparing the expression of IL-1β after UVB irradiation, followed by treatment with Centella asiatica extract, Syzygium formosum extract, or a fraction of the Syzygium formosum extract;

FIG. 9 is a graph comparing the expression of IL-8 after UVB irradiation, followed by treatment with Centella asiatica extract, Syzygium formosum extract, or a fraction of the Syzygium formosum extract;

FIG. 10 is a graph comparing the expression of COX-2 after UVB irradiation, followed by treatment with Centella asiatica extract, Syzygium formosum extract, or a fraction of the Syzygium formosum extract;

FIG. 11 is a graph showing the change in brightness of a cream containing Syzygium formosum extract or the fraction of the Syzygium formosum extract;

FIG. 12 is a graph showing the change in color of a cream containing Syzygium formosum extract or the fraction of the Syzygium formosum extract;

FIG. 13 is a graph showing the change in chromaticity of a cream containing Syzygium formosum extract or the fraction of the Syzygium formosum extract; and

FIG. 14 is a graph showing the change in viscosity of a cream containing Syzygium formosum extract or the fraction of the Syzygium formosum extract.

### DETAILED DESCRIPTION

Hereinafter, the present disclosure will be described in more detail with reference to examples, but these are merely examples and not intended to limit the scope of the present disclosure. It is obvious to those skilled in the art that the embodiments described below can be modified within the scope of the essential concept of the disclosure.

### Example 1. Preparation of Syzygium formosum Extract

Syzygium formosum leaves were harvested and dried in Nguyen Van Loung in Hanoi City, Vietnam, and 600 L of 70% (v/v) ethanol aqueous solution was added to 50 kg of dry Syzygium formosum leaves, followed by extraction at 50° C. for 24 hours. The result was filtered to obtain a first extract.

600 L of 95% (v/v) ethanol aqueous solution was added to the filtered Syzygium formosum, followed by extraction at 50° C. for 24 hours. The result was filtered to obtain a second extract. The extract was concentrated and then freeze-dried to obtain a final extract.

### Example 2-1. Preparation of First Washing Solution, Obtained by Additional Purification with Distilled Water, for the Preparation of Fraction of the Syzygium formosum Extract

2000 ml of distilled water was added to 100 g of the extract obtained in Example 1, mixed for 1 minute, and then centrifuged to recover a washing solution.

### Example 2-2. Preparation of Second Washing Solution, Obtained by Additional Purification with Distilled Water, for the Preparation of Syzygium formosum Extract

2000 ml of distilled water was added to the precipitate separated by centrifugation in Example 2-1, mixed for 1 minute, and then centrifuged to recover the washing solution.

### Example 2-3. Preparation of Third Washing Solution, Obtained by Additional Purification with Distilled Water, for the Preparation of Syzygium formosum Extract

2000 ml of distilled water was added to the precipitate separated by centrifugation in Example 2-2, mixed for 1 minute, and then centrifuged to recover the washing solution.

### Example 2-4. Preparation of Fractions of Syzygium formosum Extract Further Purified with Distilled Water

The precipitate separated by centrifugation in Example 2-3 was freeze-dried to obtain a fraction of the Syzygium formosum extract containing a great amount of triterpene.

### Experimental Example 1. Confirmation of Yield after Further Purification with Distilled Water

Table 1 shows the results of confirming the yield after further purification with distilled water.

TABLE 1

| Weight before additional | Weight after additional | |
|---|---|---|
| purification (g) | purification (g) | Yield (%) |
| 100.0 | 38.6 | 38.6 |

### Experimental Example 2. Confirmation of Changes in Amounts of Active Ingredients in the Fraction of the Syzygium formosum Extract Further Purified with Distilled Water Using LC-MS/MS

(1) LC-MS/MS Analysis Conditions

LC-MS/MS analysis was performed under the conditions shown in Table 2 and Table 3 below.

TABLE 2

| | |
|---|---|
| Device | Agilent 6470 Triple quadrupole (QQQ) |
| Column | Agilent Zorbax Eclipse plus C18 (2.1 × 100 mm) |
| Flow | 0.2 mL/min |
| Solvent | 5 mM ammonium formate in water |
| | (B) 5 mM ammonium formate in methanol |

TABLE 3

| Time (minutes) | A (%) | B (%) |
|---|---|---|
| 0 | 30 | 70 |
| 1 | 30 | 70 |
| 3 | 20 | 80 |
| 8 | 18 | 82 |
| 20 | 16 | 84 |
| 20.5 | 0 | 100 |
| 25.5 | 0 | 100 |
| 26 | 30 | 70 |
| 35 | 30 | 70 |

(2) Analysis Results

In relation to the extract obtained in Example 1 and the washing solutions and fractions obtained in Examples 2-1 to 2-4, nine active ingredients were quantified and shown in Table 4 below in ppm units. It was confirmed that the amounts of active ingredients were greater in the fraction of the Syzygium formosum extract than in the Syzygium formosum extract.

TABLE 4

| | Madecassic | Asiatic | Maslinic | Corosolic | Betulinic | Oleanolic | Ursolic | |
|---|---|---|---|---|---|---|---|---|
| | acid | acid | acid | acid | acid | acid | acid | Total |
| Example 1 | 4100 | 30900 | 7500 | 17600 | 19900 | 1400 | 4100 | 85500 |
| (extract) | | | | | | | | |
| Example 2- | 0.3 | 9.2 | 3.1 | 11.0 | 7.8 | 0.6 | 2.1 | 34.1 |
| 1 | | | | | | | | |
| Example 2- | 1.0 | 11.8 | 4.7 | 8.2 | 9.5 | 0.5 | 1.1 | 36.8 |
| 2 | | | | | | | | |
| Example 2- | 0.7 | 10.2 | 4.5 | 8.6 | 8.8 | 0.6 | 1.3 | 34.7 |
| 3 | | | | | | | | |
| Example 2- | 11600 | 106200 | 21300 | 49400 | 51600 | 4100 | 11400 | 255600 |
| 4 | | | | | | | | |
| (fraction) | | | | | | | | |

### Experimental Example 3. Confirmation of Cytotoxicity of Syzygium Formosum Extract and Fraction Thereof

HaCaT cells, a keratinocyte cell line, were treated with each of the extracts and fractions thereof prepared according to Examples 1 and 2-4, and for each case, cytotoxicity was confirmed with MTT solution after 24 hours.

Specifically, HaCaT cells were subcultured 6 to 11 times, seeded at 0.05×10⁶ cells/well in 200 ul medium (5% FBS, DMEM) in a 48 well plate and cultured overnight. The cultured cells were treated with the Syzygium formosum extract and the fraction thereof at concentrations, and then cultured at 37° C. for 24 hours. Each experiment was proceeded with an MTT solution (5 mg/ml in DPBS) in an amount of 10 ul and caused a reaction for 3 hours. After removing the medium containing the MTT solution, 300 ul of DMSO was added to the result and mixed for 5 minutes. The Syzygium formosum extract and the fraction thereof were transferred in an amount of 100 ul per well into a 96-well plate by pipetting and the cell viability (%) thereof was calculated.

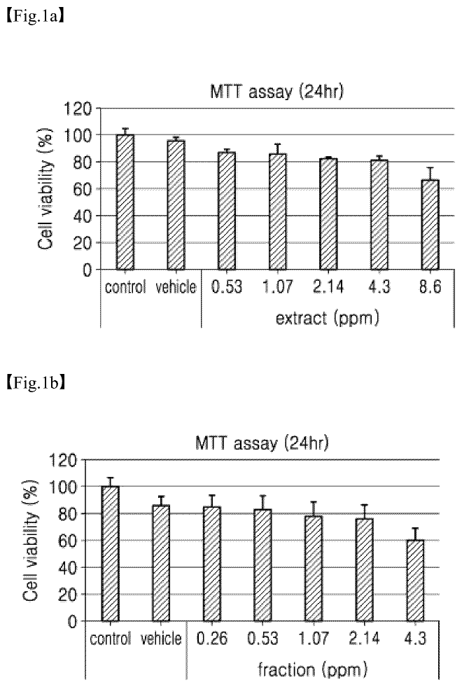

As a result, as shown in FIG. 1, in the case of Syzygium formosum extract, a cell viability of 80% or more was confirmed at a concentration of 50 ug/ml, and in the case of the fraction of the Syzygium formosum extract, a cell viability of about 80% was confirmed at a concentration of 25 ug/ml.

### Experimental Example 4. Comparison of Inflammation Inhibitory Effects of Syzygium formosum Extract and Fraction Thereof

The anti-inflammatory effect of the fraction of the Syzygium formosum extract was confirmed by identifying inflammatory cytokine expression for the inflammatory response of cells generated when the oxidative stress was given by irradiating UVB ATP.

Specifically, HaCaT cells passaged 4 times were seeded in a 6-well plate at 0.5×10⁶ cells/ml in 2 ml of medium, and pretreated with the Syzygium formosum extract and the fraction thereof for 6 hours at each concentration. A positive control was treated with vitamin C. The medium was removed and the washing process was performed twice with DPBS. With 500 ul of DPBS therein, 20 mJ of UVB was irradiated for 30 seconds. DPBS was removed, and 2 ml of DMEM serum-free medium, in which 15 ug/ml of the Syzygium formosum extract and the fraction thereof were dissolved, was placed, followed by 18 hours of culturing. Then, RNA was extracted and inflammatory cytokine expression was confirmed by a polymerase chain reaction test (PCR).

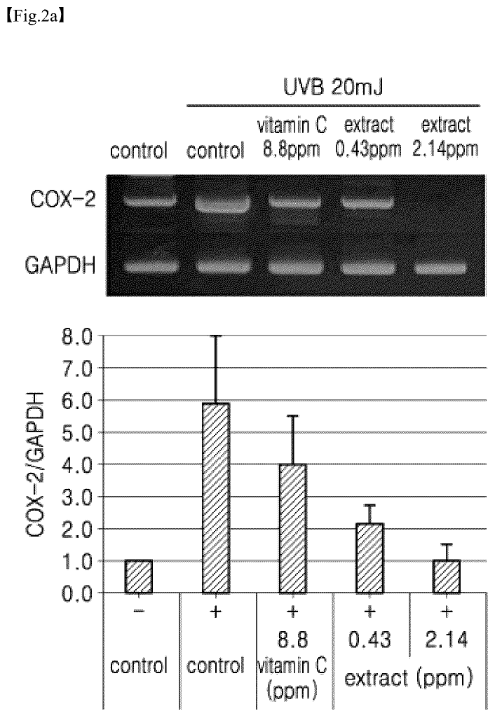

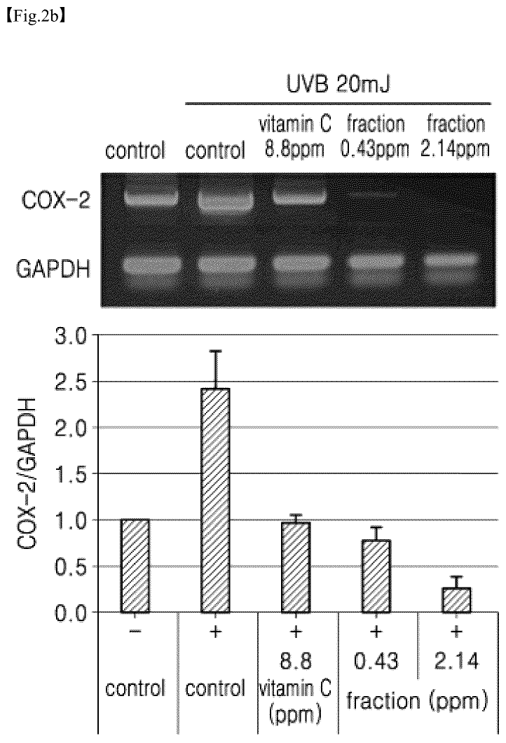

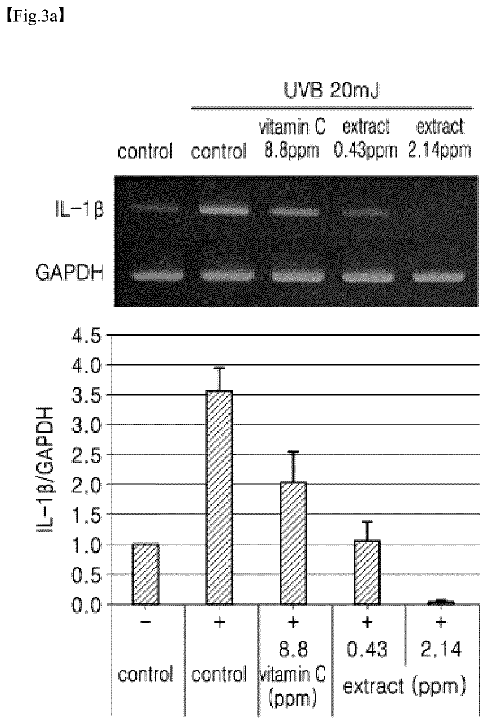

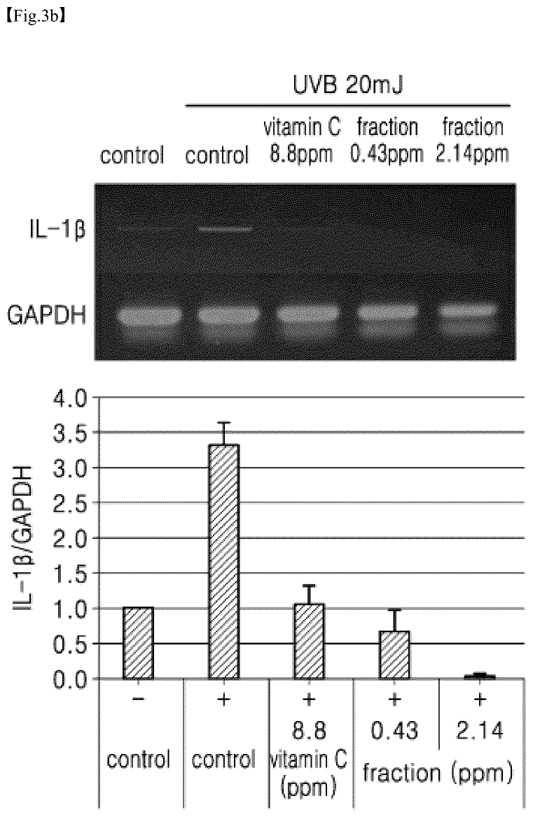

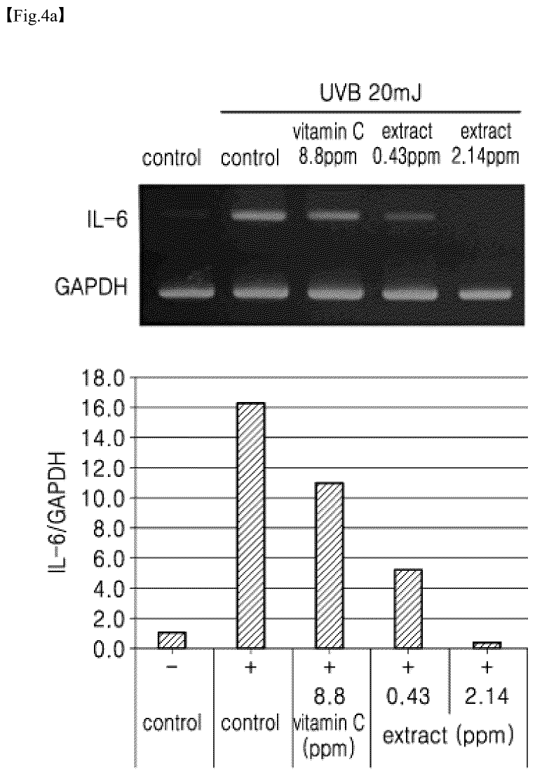

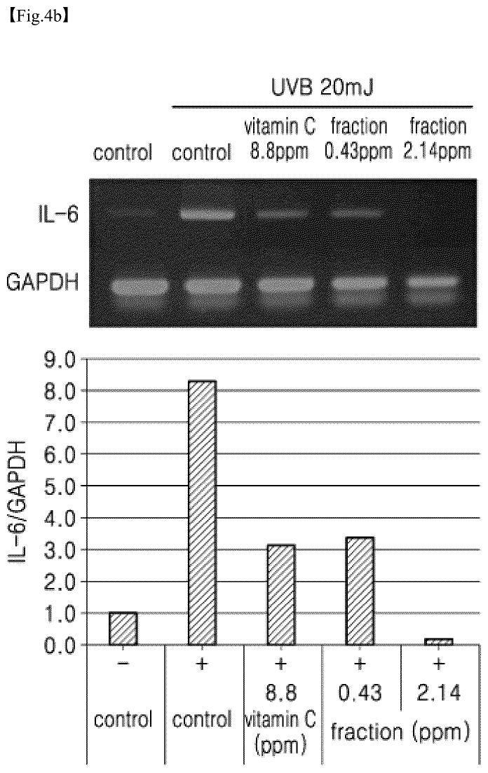

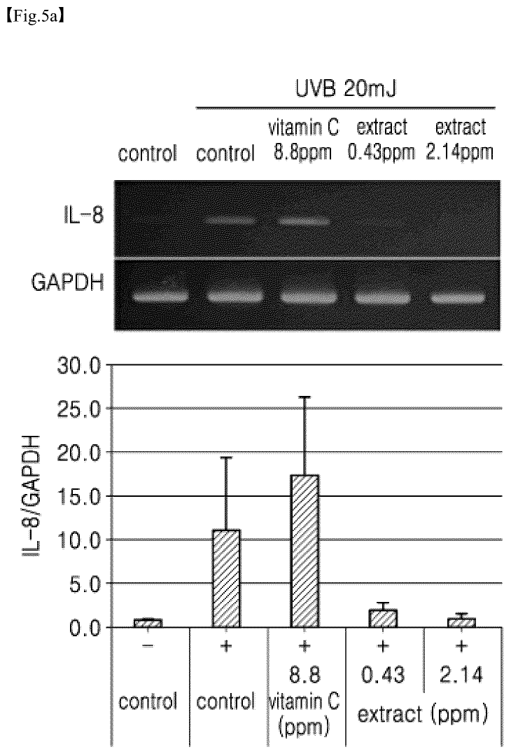

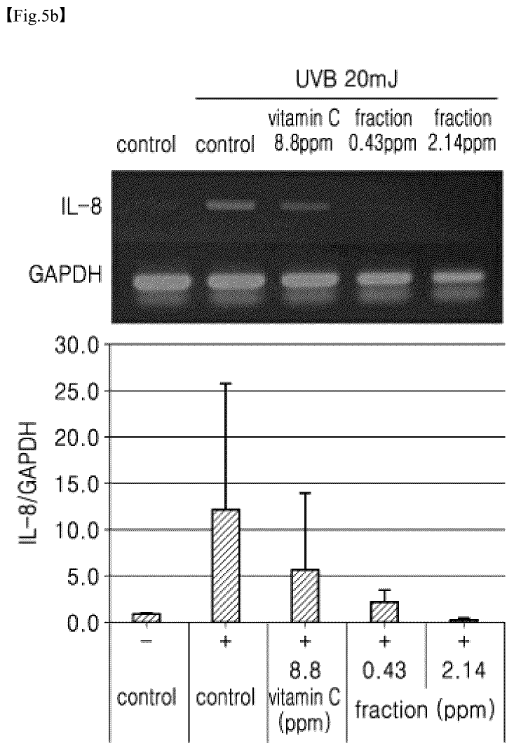

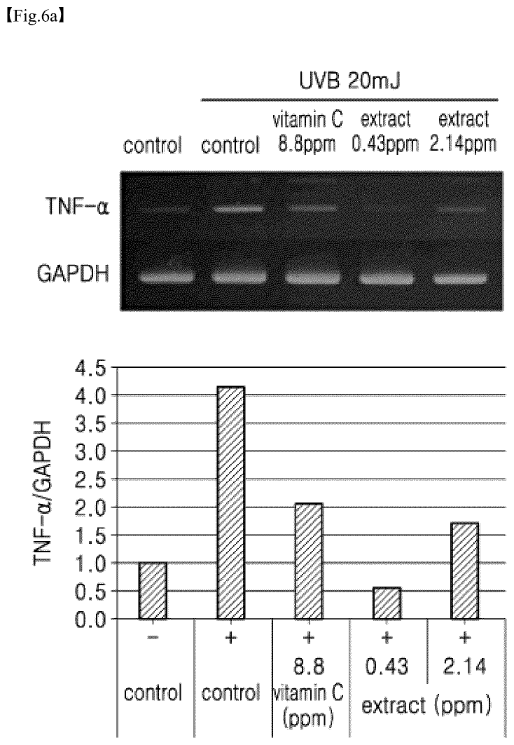

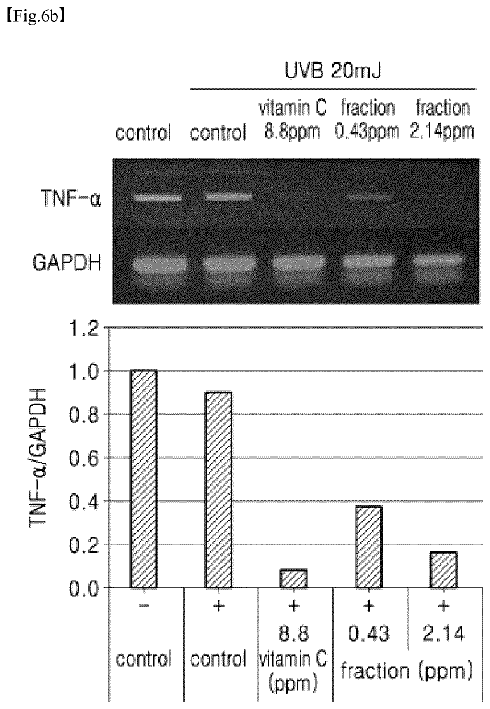

As a result, as shown in FIGS. 2 to 6, it was confirmed that the fraction of the Syzygium formosum extract more effectively inhibits inflammatory cytokine expression than vitamin C and the Syzygium formosum extract.

### Experimental Example 5. Comparison of Active Ingredient Amounts of Fraction of the Syzygium formosum Extract and Centella Asiatica Extract

Active ingredients of the fraction of the Syzygium formosum extract and two types of Centella asiatica extracts were compared.

Specifically, extracts and fractions were prepared in the same manner as in Example 1 and Examples 2-1 to 2-4 except that 40 ml of 70% ethanol was added to 2 g of dry Syzygium formosum leaves. Separately, 40 ml of 70% ethanol was added to each of two kinds of Centella asiatica, each having an amount of 2 g. The extraction process was performed 50 OC for 24 hours to collect an extract solution. Then, LC-MS/MS analysis was performed under the conditions shown in Tables 2 and 3 to quantify 9 active ingredients, and the amounts of the active ingredients are shown in Table 5 below in ppm units.

As a result, it was confirmed that the fraction of the Syzygium formosum extract had greater active ingredient amounts compared to Syzygium formosum extract.

TABLE 5

| | Madecassic | Asiatic | Maslinic | Corosolic | Betulinic | Oleanolic | Ursolic | |
|---|---|---|---|---|---|---|---|---|
| | acid | acid | acid | acid | acid | acid | acid | Total |
| Syzygium | 49.1 ± 3 | 629.5 ± 35.5 | 51.9 ± 3.4 | 117 ± 2.9 | 263.5 ± 25.4 | 16.2 ± 1 | 40.6 ± 2.7 | 1235.2 ± 74.9 |
| formosum | | | | | | | | |
| extract | | | | | | | | |
| First | 14.4 ± 0.2 | 340.6 ± 32.5 | 47.4 ± 1.7 | 191.3 ± 21.1 | 153.3 ± 31.5 | 11.1 ± 0.1 | 32.7 ± 1.6 | 801.2 ± 81.4 |
| washing | | | | | | | | |
| solution of | | | | | | | | |
| Syzygium | | | | | | | | |
| formosum | | | | | | | | |
| extract | | | | | | | | |
| Second | 19.4 ± 1.7 | 225.2 ± 56.1 | 41.8 ± 3.2 | 200.7 ± 49.5 | 294.3 ± 55 | 9.4 ± 1 | 30.1 ± 2.3 | 829.4 ± 169.6 |
| washing | | | | | | | | |
| solution of | | | | | | | | |
| Syzygium | | | | | | | | |
| formosum | | | | | | | | |
| extract | | | | | | | | |
| Third | 13.2 ± 1.1 | 387.6 ± 42.7 | 43 ± 0.4 | 183 ± 33.4 | 134.8 ± 17.7 | 9.4 ± 0.2 | 27.9 ± 0 | 806 ± 92.3 |
| washing | | | | | | | | |
| solution of | | | | | | | | |
| Syzygium | | | | | | | | |
| formosum | | | | | | | | |
| extract | | | | | | | | |
| Fraction of | 41 ± 0.8 | 503.1 ± 6 | 107.9 ± 1.1 | 310.5 ± 1 | 302.4 ± 15.6 | 26.5 ± 0.2 | 67.6 ± 1.1 | 1376.6 ± 17.4 |
| Syzygium | | | | | | | | |
| formosum | | | | | | | | |
| extract | | | | | | | | |
| Centella | 102.0 ± 22.4 | 190.4 ± 59.7 | N/D | N/D | N/D | N/D | N/D | 292.9 ± 82.8 |
| asiatica 1 | | | | | | | | |
| Centella | 22.3 ± 0.6 | 38.4 ± 1.1 | N/D | N/D | N/D | N/D | N/D | 60.7 ± 0.5 |
| asiatica 2 | | | | | | | | |

### Experimental Example 6. Comparison of Inflammation Inhibitory Effects of Centella Asiatica Extract, and Syzygium formosum Extract and Fraction Thereof

The anti-inflammatory effect of the fraction of the Syzygium formosum extract was confirmed by identifying inflammatory cytokine expression for the inflammatory response of cells generated when the oxidative stress was given by irradiating UVB ATP.

Specifically, HaCaT cells passaged 16 times were seeded in a 6-well plate at 0.5×10⁶ cells/ml in 2 ml of medium, and pretreated with Centella asiatica extract, the Syzygium formosum extract and the fraction thereof for 6 hours at each concentration. The medium was removed and the washing process was performed twice with DPBS. With 500 ul of DPBS therein, 20 mJ of UVB was irradiated for 30 seconds. DPBS was removed, and 2 ml of DMEM serum-free medium with 8.8 ppm of vitamin C, 0.43 ppm of the Syzygium formosum extract, and 2.14 ppm of the fraction of the Syzygium formosum extract, each dissolved therein, was placed, followed by 18 hours of culturing. Then, RNA was extracted and inflammatory cytokine expression was confirmed by a polymerase chain reaction test (PCR).

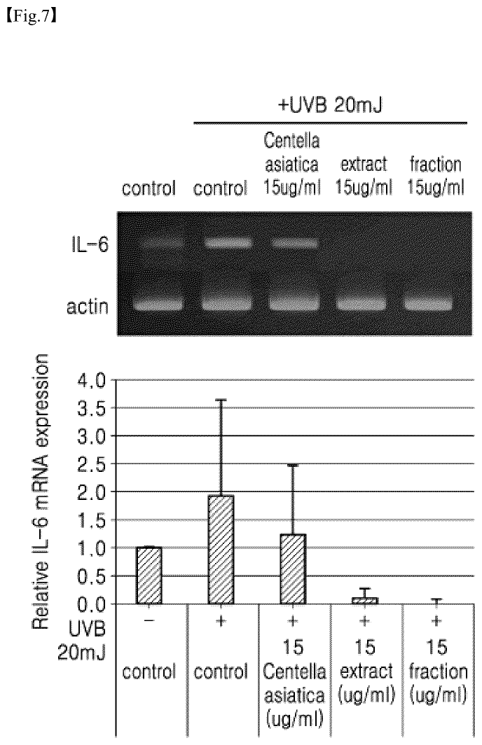

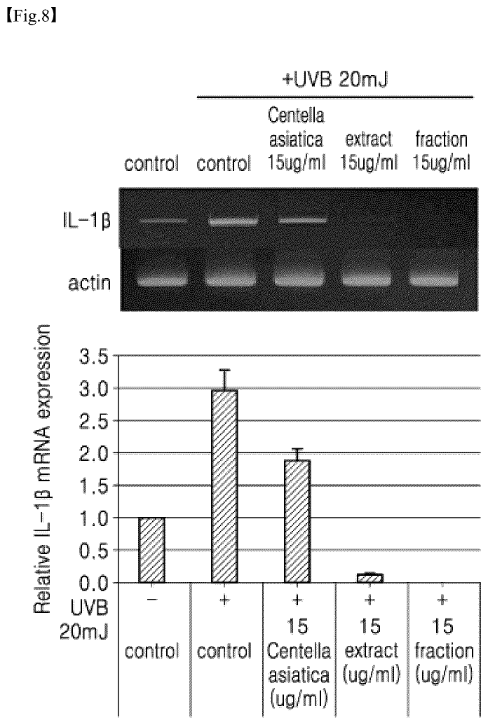

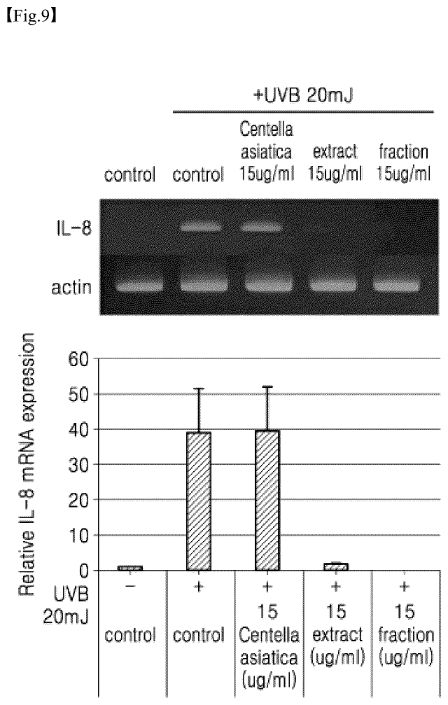

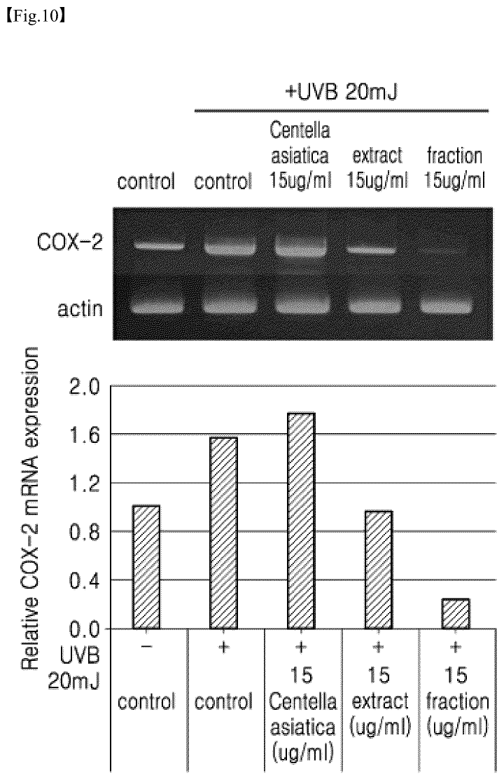

As a result, as shown in FIGS. 7 to 10, it was confirmed that the fraction of the Syzygium formosum extract more effectively inhibits inflammatory cytokine expression than the Centella asiatica extract and the Syzygium formosum extract. These results indicate that fraction of the Syzygium formosum extract has a higher inhibitory effect on inflammation than the Centella asiatica extract and the Syzygium formosum extract.

### Preparation Example 1. Preparation of Cream Containing Syzygium Formosum Extract

In order to check the cosmetic material properties of Syzygium formosum extract, a cream was prepared by a conventional method according to the composition shown in Table 6 below using the extract obtained in Example 1. The amounts of the extract and the fraction obtained in Example 1 and Example 2-4 were adjusted in preparing a cream due to the difference in the amounts of active ingredients of the extract and the fraction.

TABLE 6

| Source Name | Function | Amount (wt %) |
|---|---|---|
| Extract obtained in Example 1 | Functional | 0.3 |
| | component | |
| Cetearyl alcohol | Emollient | 5.00 |
| Cetyl palmitate | Emollient | 2.00 |
| Glyceryl stearate | Emulsifier | 2.00 |
| Beeswax | Emollient | 1.00 |
| Polyglyceryl-3methylglucose | Emulsifier | 2.00 |
| distearate | | |
| Squalane | Emollient | 5.00 |
| Cetylethylhexanoate | Emollient | 3.00 |
| Caprylic/capric triglyceride | Emollient | 3.00 |
| 1,2-hexanediol | Humectant | 3.00 |
| Dipropylene glycol | Humectant | 3.00 |
| Glycerin | Humectant | 5.00 |
| Xanthan gum | Thickener | 0.10 |
| Ammonium | Thickener | 0.30 |
| acryloyldimethyltaurate/VP | | |
| Copolymer | | |
| Fragrance | Fragrance | Appropriate amount |
| Purified water | Solvent | to 100 |

### Preparation Example 2. Preparation of a Cream Containing Fraction of the Syzygium formosum Extract

In order to check the cosmetic material properties of the fraction of the Syzygium formosum extract, a cream was prepared by a conventional method according to the composition shown in Table 7 below using the fractions obtained in Examples 2-4, and the amounts of the extract and the fraction obtained in Example 1 and Example 2-4 were adjusted in preparing a cream due to the difference in the amounts of active ingredients of the extract and the fraction.

TABLE 7

| Source Name | Function | Amount (wt %) |
|---|---|---|
| Washing solution or fraction | Functional | 0.1 |
| obtained in Examples 2 to 4 | component | |
| Cetearyl alcohol | Emollient | 5.00 |
| Cetyl palmitate | Emollient | 2.00 |
| Glyceryl stearate | Emulsifier | 2.00 |
| Beeswax | Emollient | 1.00 |
| Polyglyceryl-3methylglucose | Emulsifier | 2.00 |
| distearate | | |
| Squalane | Emollient | 5.00 |
| Cetylethylhexanoate | Emollient | 3.00 |
| Caprylic/capric triglyceride | Emollient | 3.00 |
| 1,2-hexanediol | Humectant | 3.00 |
| Dipropylene glycol | Humectant | 3.00 |
| Glycerin | Humectant | 5.00 |
| Xanthan gum | Thickener | 0.10 |
| Ammonium | Thickener | 0.30 |
| acryloyldimethyltaurate/VP | | |
| Copolymer | | |
| Fragrance | Fragrance | Appropriate amount |
| Purified water | Solvent | to 100 |

### Experimental Example 3. Confirmation of Cosmetic Material Properties of Fractions of Syzygium formosum Extract Through Accelerated Testing

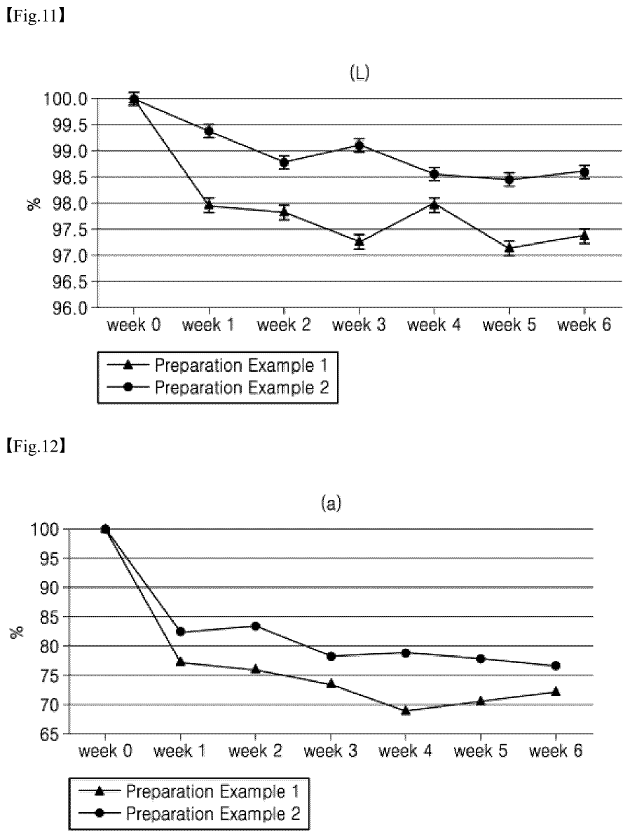

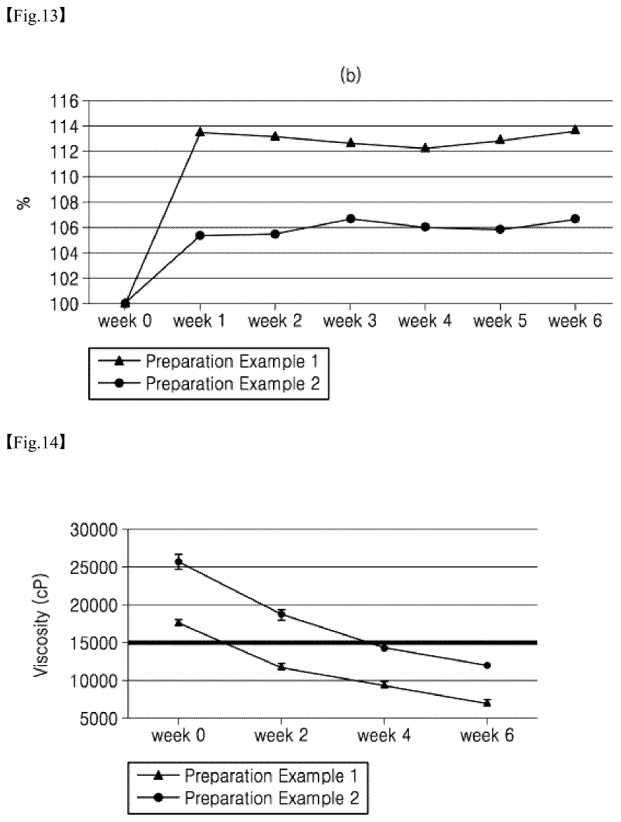

The cream prepared in Preparation Example 1 was stored at 45° C. for 6 weeks, and the pH and chromaticity thereof were measured every week and shown in Table 8, and FIGS. 11 to 13 are expressed as a percentage (%) on the basis of the measurement of brightness (L*), color (A), and chromaticity (B) values at week 0. The viscosity measurement results are shown in FIG. 14, and the viscosity of a typical cream is indicated by a red line.

As a result, as shown in FIGS. 11 to 14, the cream containing the fraction of the Syzygium formosum extract maintained a viscosity of about 5000 cP higher than that of the cream containing Syzygium formosum extract, and the brightness (L) of the former was higher and changed less than the latter. In addition, it was confirmed that the cream containing the fraction of the Syzygium formosum extract changed less in aspects of color (a) and chromaticity (b). Accordingly, it was confirmed that the formulation was more stable.

TABLE 8

| | Week | Week | Week | Week | Week | Week | Week |
|---|---|---|---|---|---|---|---|
| | 0 | 1 | 2 | 3 | 4 | 5 | 6 |
| PH of the | 6.35 | 6.24 | 6.04 | 6.05 | 6.02 | 6.01 | 5.96 |
| cream | | | | | | | |
| containing | | | | | | | |
| the extract | | | | | | | |
| obtained in | | | | | | | |
| Preparation | | | | | | | |
| Example 1 | | | | | | | |
| PH of the | 6.64 | 6.47 | 6.27 | 6.29 | 6.27 | 6.21 | 6.15 |
| cream | | | | | | | |
| containing | | | | | | | |
| the fraction | | | | | | | |
| obtained in | | | | | | | |
| Preparation | | | | | | | |
| Example 1 | | | | | | | |

A composition including a fraction of the Syzygium formosum extract according to one aspect as an active ingredient can be usefully used in the prevention, amelioration, or treatment of inflammatory diseases by inhibiting the expression of inflammatory cytokines, and cosmetics including the same has high stability.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

1. A method of ameliorating treating skin inflammation, comprising administering a fraction of Syzygium formosum extract to an individual in need thereof.

2. (canceled)

3. The method of claim 1, wherein the fraction is a hexane fraction, an ethyl acetate fraction, a butanol fraction, or a water fraction of the Syzygium formosum extract.

4. The method of claim 1, wherein the fraction is obtained by repeating a fractioning process 1 to 5 times.

5. The method of claim 1, further comprising an emollient, a humectant, and a thickener.

6. The method of claim 5, wherein the emollient is selected from cetearyl alcohol, cetyl palmitate, beeswax, squalane, cetylethylhexanoate and caprylic/capric triglyceride.

7. The method of claim 5, wherein the humectant is selected from 1,2-hexanediol, dipropylene glycol, and glycerin.

8. The method of claim 5, wherein the thickener is selected from xanthan gum and an ammonium acryloyldimethyltaurate/VP copolymer.

9-11. (canceled)

12. The method of claim 1, wherein the skin inflammatory selected from skin wounds, dermatitis, atopic dermatitis, pruritus, eczema skin disease, dry eczema, erythema, urticaria, psoriasis, weak rash, and acne.

13-14. (canceled)
